(19) United States
(12) Patent Application Publication
(10) Pub. No.: US 2026/0226858 A1
(43) Pub. Date: Aug. 6, 2026

(54) EXHAUST MONITORING APPARATUS

(71) Applicant: TOYOTA JIDOSHA KABUSHIKI KAISHA, Toyota-shi (JP)

(72) Inventors: Yuji HANO, Toyota-shi (JP); Atsushi MORIKAWA, Toyota-shi (JP)

(73) Assignee: TOYOTA JIDOSHA KABUSHIKI KAISHA, Toyota-shi (JP)

(21) Appl. No.: 19/455,305

(22) Filed: Jan. 21, 2026

(30) Foreign Application Priority Data
Jan. 31, 2025 (JP) 2025-014819

(51) Int. Cl. F01N 11/00

(52) U.S. Cl. CPC F01N 11/00; F01N 2560/026; F01N 2560/07; F01N 2900/08; F01N 2900/1402; F01N 2900/1411

(57) ABSTRACT

An exhaust monitoring apparatus sets a transport delay time spanning from when air passes through a portion of an intake passage where an air flow meter is provided to when exhaust gas derived from the air that has passed through the air flow meter reaches a nitrogen oxide sensor. The exhaust monitoring apparatus estimates an emission amount of nitrogen oxides based on an output value of the nitrogen oxide sensor and an output value of the air flow meter that was output earlier than a point in time at which the nitrogen oxide sensor output the output value by the transport delay time.

### CROSS-REFERENCE TO RELATED APPLICATIONS

This application is based upon and claims the benefit of priority from Japanese Patent Application No. 2025-014819, filed on Jan. 31, 2025, the entire contents of which are incorporated herein by reference.

### BACKGROUND

### 1. Field

The present disclosure relates to an exhaust monitoring apparatus.

### 2. Description of Related Art

JPH7-63096A discloses an exhaust monitoring apparatus for a vehicle.

The vehicle includes an intake passage of the engine that is provided with an air flow meter. The vehicle includes an exhaust passage provided with an exhaust purification device that removes nitrogen oxides (NOx) contained in exhaust gas. The vehicle includes a nitrogen oxide sensor, which measures the concentration of NOx contained in exhaust gas, on the downstream side of the exhaust purification device in the exhaust passage.

The exhaust monitoring apparatus estimates an amount of NOx emitted for a predetermined period of time based on an output value of the air flow meter and an output value of the nitrogen oxide sensor that were output at the same time.

In order to estimate the emission amount of NOx more accurately, there is room for improvement in the manner of estimation performed by the exhaust monitoring apparatus.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

An aspect of the present disclosure provides an exhaust monitoring apparatus. The exhaust monitoring apparatus is mounted on a vehicle including an exhaust purification device that is disposed in an exhaust passage of an engine. The exhaust purification device is configured to remove nitrogen oxides contained in exhaust gas. The exhaust monitoring apparatus includes a nitrogen oxide sensor disposed downstream of the exhaust purification device in the exhaust passage and configured to measure a concentration of nitrogen oxides contained in exhaust gas, an air flow meter disposed in an intake passage of the engine and configured to measure an amount of air flowing through the intake passage, and processing circuitry configured to set a transport delay time. The transport delay time spans from when air passes through a portion of the intake passage where the air flow meter is provided to when exhaust gas derived from the air that has passed through the air flow meter reaches the nitrogen oxide sensor. The processing circuitry is further configured to estimate an emission amount of nitrogen oxides based on an output value of the nitrogen oxide sensor and an output value of the air flow meter that was output earlier than a point in time at which the nitrogen oxide sensor output the output value by the transport delay time.

Other features and aspects will be apparent from the following detailed description, the drawings, and the claims.

### BRIEF DESCRIPTION OF THE DRAWINGS

FIG. 1 is a schematic diagram showing the configuration of a vehicle including an exhaust monitoring apparatus according to an embodiment.

FIG. 2 is a timing diagram illustrating changes in the output value of the air flow meter and changes in the output value of the nitrogen oxide sensor.

FIG. 3 is a flowchart illustrating a series of processes executed when the exhaust monitoring apparatus shown in FIG. 1 acquires the output value of the air flow meter.

FIG. 4 is a diagram illustrating how the exhaust monitoring apparatus shown in FIG. 1 sets the transport delay time.

FIG. 5 is a flowchart illustrating a series of processes executed when the exhaust monitoring apparatus shown in FIG. 1 acquires the output value of the nitrogen oxide sensor.

FIG. 6 is a diagram illustrating how the exhaust monitoring apparatus shown in FIG. 1 executes a search process.

FIG. 7 is a diagram illustrating how the exhaust monitoring apparatus shown in FIG. 1 estimates the volume of exhaust gas.

FIG. 8 is a flowchart illustrating a series of processes executed when the exhaust monitoring apparatus shown in FIG. 1 estimates the amount of nitrogen oxides emitted from the vehicle.

FIG. 9 is a diagram illustrating how the exhaust monitoring apparatus according to a first modification sets the transport delay time.

FIG. 10 is a diagram illustrating how the exhaust monitoring apparatus according to a second modification sets the transport delay time.

FIG. 11 is a diagram illustrating how the exhaust monitoring apparatus according to a third modification sets the transport delay time.

FIG. 12 is a diagram illustrating how the exhaust monitoring apparatus according to a fourth modification sets the transport delay time.

FIG. 13 is a diagram illustrating how the exhaust monitoring apparatus according to a fifth modification sets the transport delay time.

FIG. 14 is a diagram illustrating how the exhaust monitoring apparatus according to a sixth modification sets the transport delay time.

FIG. 15 is a diagram illustrating how the exhaust monitoring apparatus according to a seventh modification sets the transport delay time.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

This description provides a comprehensive understanding of the methods, apparatuses, and/or systems described. Modifications and equivalents of the methods, apparatuses, and/or systems described are apparent to one of ordinary skill in the art. Sequences of operations are exemplary, and may be changed as apparent to one of ordinary skill in the art, with the exception of operations necessarily occurring in a certain order. Descriptions of functions and constructions that are well known to one of ordinary skill in the art may be omitted.

Exemplary embodiments may have different forms, and are not limited to the examples described. However, the examples described are thorough and complete, and convey the full scope of the disclosure to one of ordinary skill in the art.

In this specification, “at least one of A and B” should be understood to mean “only A, only B, or both A and B.”

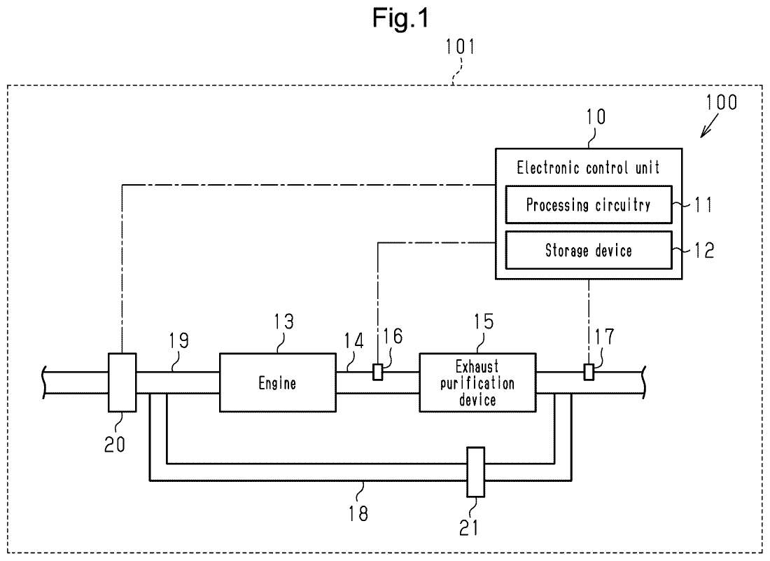

An embodiment of an exhaust monitoring apparatus will now be described with reference to FIGS. 1 to 8.

### Configuration of Vehicle 101

As shown in FIG. 1, a vehicle 101 includes an engine 13, an exhaust passage 14, an exhaust purification device 15, and an intake passage 19.

The exhaust passage 14 is connected to the engine 13. The exhaust gas discharged from the engine 13 is discharged out of the vehicle 101 through the exhaust passage 14.

The exhaust purification device 15 is provided at a position along the exhaust passage 14. The exhaust purification device 15 removes nitrogen oxides (NOx) contained in exhaust gas. Examples of the exhaust purification device 15 include a three-way catalytic converter that removes, for instance, NOx, using a three-way catalyst. Examples of the exhaust purification device 15 may include a selective catalytic reduction catalyst that reduces NOx in exhaust gas, using ammonia as a reducing agent.

The intake passage 19 is connected to the engine 13. The intake passage 19 is open to the outside of the vehicle 101. When the engine 13 intakes air, ambient air outside the vehicle 101 flows through the intake passage 19 into the combustion chamber of the engine 13.

The vehicle 101 includes a recirculation passage 18 and an exhaust gas recirculation (EGR) valve 21.

The recirculation passage 18 is connected to a portion of the exhaust passage 14 downstream of the exhaust purification device 15 and a portion of the intake passage 19 downstream of the air flow meter 20. The air flow meter 20 will be described later.

Part of the exhaust gas that has passed through the exhaust purification device 15 in the exhaust passage 14 flows into the recirculation passage 18. The exhaust gas that has passed through the recirculation passage 18 flows into the intake passage 19, and merges with the air that has flowed into the intake passage 19 from the outside of the vehicle 101. In this manner, the recirculation passage 18 recirculates exhaust gas to the intake passage 19.

The EGR valve 21 is provided at a position along the recirculation passage 18. The EGR valve 21 regulates the amount of exhaust gas recirculated through the recirculation passage 18 to the intake passage 19.

The vehicle 101 includes an exhaust monitoring apparatus 100. The exhaust monitoring apparatus 100 estimates the amount of NOx emitted from the vehicle 101. The emission amount of NOx estimated by the exhaust monitoring apparatus 100 represents the mass of NOx emitted from the vehicle 101 per unit time.

The exhaust monitoring apparatus 100 includes an electronic control unit 10, an air-fuel ratio sensor 16, a nitrogen oxide sensor 17, and an air flow meter 20.

The electronic control unit 10 includes processing circuitry 11 and a storage device 12. The storage device 12 stores program codes or instructions configured to cause the CPU to execute the processes. The storage device 12, or a computer-readable medium, includes any type of medium that is accessible by general-purpose computers and dedicated computers. The processing circuitry 11 executes programs stored in the storage device 12 to execute various processes. The processing circuitry 11 includes a processor. The processing circuitry 11 may include one or more dedicated hardware circuits, such as application-specific integrated circuits (ASICs), that execute at least some of various processes. Alternatively, the processing circuitry 11 may include a combination of the processor and the dedicated hardware circuitry. The processor includes a CPU and a memory, such as a RAM and a ROM.

The air-fuel ratio sensor 16 is disposed upstream of the exhaust purification device 15 in the exhaust passage 14. The air-fuel ratio sensor 16 measures the air-fuel ratio of the exhaust gas prior to passing through the exhaust purification device 15.

The nitrogen oxide sensor 17 is disposed downstream of the exhaust purification device 15 in the exhaust passage 14. The nitrogen oxide sensor 17 measures the concentration of NOx contained in the exhaust gas that has passed through the exhaust purification device 15.

The air flow meter 20 is provided at a position along the intake passage 19. The air flow meter 20 measures the amount of air flowing through the intake passage 19. The air flow meter 20 measures, as the amount of air flowing through the intake passage 19, the mass flow rate of the air flowing through the intake passage 19 per unit time. The air flow meter 20 may measure, as the amount of air flowing through the intake passage 19, the volumetric flow rate of the air flowing through the intake passage 19 per unit time.

The electronic control unit 10 is communicably connected to the air-fuel ratio sensor 16. The electronic control unit 10 periodically acquires an output value from the air-fuel ratio sensor 16.

The electronic control unit 10 is communicably connected to the nitrogen oxide sensor 17. The electronic control unit 10 periodically acquires an output value from the nitrogen oxide sensor 17.

The electronic control unit 10 is communicably connected to the air flow meter 20. The electronic control unit 10 periodically acquires an output value from the air flow meter 20.

### Outline of Transport Delay Time TL

The exhaust monitoring apparatus 100 estimates the amount of NOx emitted from the exhaust passage 14, based on the output value of the air flow meter 20 and the output value of the nitrogen oxide sensor 17. The electronic control unit 10 periodically acquires output values from the air flow meter 20 and the nitrogen oxide sensor 17. The exhaust monitoring apparatus 100 estimates the emission amount of NOx each time the electronic control unit 10 acquires the output values of the air flow meter 20 and the nitrogen oxide sensor 17. Hereinafter, the emission amount of NOx output by the exhaust monitoring apparatus 100 each time the output values of the air flow meter 20 and the nitrogen oxide sensor 17 are acquired, will be referred to as an instantaneous emission amount.

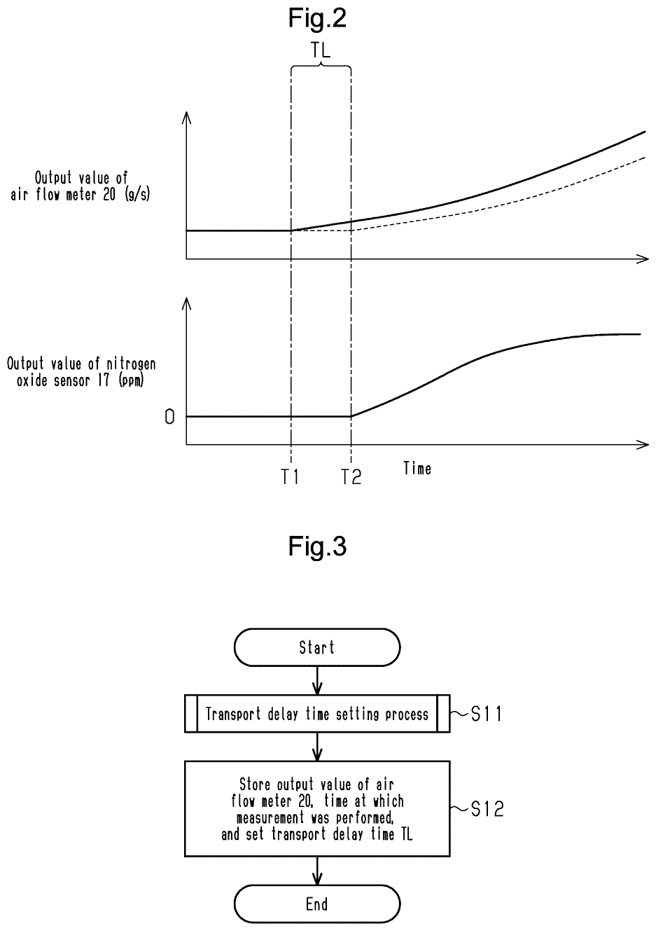

In the upper graph of FIG. 2, the solid line indicates changes in the output value of the air flow meter 20 in a hypothetical case. The air flow meter 20 outputs, as the amount of air flowing through the intake passage 19, the mass flow rate of the air flowing through the intake passage 19 per unit time. The lower graph in FIG. 2 illustrates an example of changes in the output value of the nitrogen oxide sensor 17 in the hypothetical case.

In the hypothetical case, the engine 13 starts operating at time T1. After the engine 13 starts operating, the amount of air taken in by the engine 13 gradually increases.

When the amount of air taken in by the engine 13 increases, the amount of air flowing through the intake passage 19 increases. As indicated by the solid line in the upper graph in FIG. 2, the output value of the air flow meter 20 increases from time T1.

After the engine 13 starts operating, exhaust gas is generated based on the intake air. Thus, the output value of the nitrogen oxide sensor 17 is larger than zero. As shown in the lower graph of FIG. 2, the output value of the nitrogen oxide sensor 17 is larger than zero from time T2.

In the hypothetical case, the time at which the output value of the air flow meter 20 starts rising is different from the time at which the nitrogen oxide sensor 17 starts outputting a value larger than zero.

As shown in FIG. 1, the air flow meter 20 is provided in the intake passage 19. The nitrogen oxide sensor 17 is provided in the exhaust passage 14. Air drawn into the vehicle 101 passes through a portion of the intake passage 19 where the air flow meter 20 is provided, and is then burned by the engine 13 to form exhaust gas. The exhaust gas discharged from the engine 13 passes through the exhaust passage 14 and reaches a portion where the nitrogen oxide sensor 17 is provided.

The air that has passed through the portion where the air flow meter 20 is provided does not immediately reach the portion where the nitrogen oxide sensor 17 is provided in the form of exhaust gas. Accordingly, the air that has passed through the portion where the air flow meter 20 is provided requires a certain amount of time to form exhaust gas and reach the portion where the nitrogen oxide sensor 17 is provided. In the hypothetical case, the air that has passed through the portion where the air flow meter 20 is provided at time T1 reaches the portion where the nitrogen oxide sensor 17 is provided in the form of exhaust gas at time T2.

Before the air measured by the air flow meter 20 forms exhaust gas and reaches the portion where the nitrogen oxide sensor 17 is provided, a time lag occurs. Thus, if the exhaust monitoring apparatus 100 uses the output value of the air flow meter 20 and the output value of the nitrogen oxide sensor 17 that were output at the same time, the exhaust monitoring apparatus 100 cannot accurately estimate the amount of NOx emitted from the vehicle 101.

To solve this problem, the exhaust monitoring apparatus 100 estimates the amount of NOx emitted from the vehicle 101 based on the output value of the nitrogen oxide sensor 17 and the output value of the air flow meter 20 that were output at different times. The output value of the air flow meter 20 is a value that was output earlier than the point in time at which the nitrogen oxide sensor 17 output the output value by the transport delay time TL. The transport delay time TL spans from when air passes through the portion of the intake passage 19 where the air flow meter 20 is provided to when exhaust gas derived from the air that has passed through the air flow meter 20 reaches the nitrogen oxide sensor 17.

FIG. 2 illustrates the transport delay time TL. In FIG. 2, the transport delay time TL indicates the length of time from T1 to T2.

In the upper graph of FIG. 2, the changes in the output value of the air flow meter 20 reflecting the transport delay time TL are shown by the dotted line. In the upper graph of FIG. 2, the changes in the output value indicated by the dotted line are obtained by shifting the changes in the output value indicated by the solid line in the positive direction along the horizontal axis by the transport delay time TL. In the graph on the upper side of FIG. 2, the value at time T2 on the dotted line is the value at time T1 on the solid line. The exhaust monitoring apparatus 100 treats the output value of the air flow meter 20 that was output earlier than the current time by the transport delay time TL, as the output value of the air flow meter 20 that was output earlier than the point in time at which the nitrogen oxide sensor 17 output the output value by the transport delay time TL.

The exhaust monitoring apparatus 100 estimates the amount of NOx emitted from the vehicle 101, based on the output value of the air flow meter 20 indicated by the dotted line in the upper graph of FIG. 2 and the output value of the nitrogen oxide sensor 17 indicated by the lower graph of FIG. 2. For example, the exhaust monitoring apparatus 100 estimates the instantaneous emission amount based on the value at time T2 on the dotted line of the upper graph in FIG. 2 and the value at time T2 of the lower graph in FIG. 2. In this case, the exhaust monitoring apparatus 100 estimates the instantaneous emission amount at T2 based on the value at time T1 on the solid line of the upper graph of FIG. 2 and the value at time T2 in the lower graph of FIG. 2.

### Processes When the Output Value of the Air Flow Meter 20 is Acquired

FIG. 3 illustrates a series of processes executed by the exhaust monitoring apparatus 100. The series of processes illustrated in FIG. 3 is executed by the processing circuitry 11 of the electronic control unit 10. Upon acquiring an output value from the air flow meter 20, the processing circuitry 11 starts executing the series of processes illustrated in FIG. 3. In addition to the output value of the air flow meter 20, the processing circuitry 11 acquires information indicating the time at which the air flow meter 20 performed measurement.

Hereinafter, in FIGS. 3, 5, and 8, S denotes a step.

Upon starting the series of processes illustrated in FIG. 3, the processing circuitry 11 first executes the process of S11. In the process of S11, the processing circuitry 11 executes a transport delay time setting process. The transport delay time setting process sets a proper transport delay time TL for estimating the instantaneous emission amount based on the output value of the air flow meter 20.

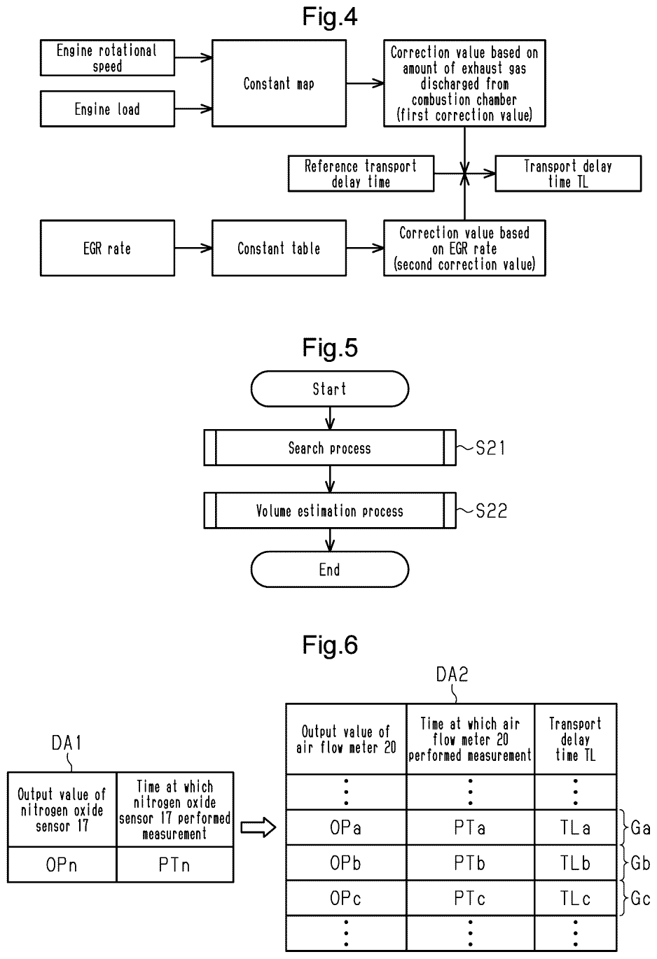

The outline of the transport delay time setting process will now be described with reference to FIG. 4.

FIG. 4 illustrates how the processing circuitry 11 sets the transport delay time TL during the transport delay time setting process.

The processing circuitry 11 calculates the transport delay time TL by correcting a reference transport delay time. The reference transport delay time is a reference time that has been set for calculating the transport delay time TL. The reference transport delay time is measured in advance by, for example, an experiment under a specific condition.

Hereinafter, the expression “discharge amount of exhaust gas” may be used. The discharge amount of exhaust gas refers to the volume of exhaust gas.

The amount of exhaust gas discharged from the combustion chamber of the engine 13 affects the transport delay time TL. The larger the amount of exhaust gas discharged from the combustion chamber, the higher the flow speed of the exhaust gas flowing through the exhaust passage 14. As a result, the transport delay time TL decreases. The smaller the amount of exhaust gas discharged from the combustion chamber, the lower the flow speed of the exhaust gas flowing through the exhaust passage 14. As a result, the transport delay time TL increases.

The processing circuitry 11 corrects the reference transport delay time based on the amount of exhaust gas discharged from the combustion chamber.

The amount of exhaust gas discharged from the combustion chamber is determined based on an engine rotational speed, which is the rotational speed of the output shaft of the engine 13, and an engine load. The engine load refers to the ratio of the volume of air drawn in one cycle to the stroke volume per cycle of the engine 13.

The processing circuitry 11 acquires information related to the engine rotational speed from, for example, a sensor that measures the engine rotational speed. For example, the processing circuitry 11 estimates the engine load, based on the information related to the engine rotational speed and the output value of the air flow meter 20.

The exhaust monitoring apparatus 100 stores, in the storage device 12, a constant map used to output a correction value that is based on the amount of exhaust gas discharged from the combustion chamber. The correction value based on the amount of exhaust gas discharged from the combustion chamber may be referred to as a first correction value. The constant map is used to output a correction value corresponding to a combination of the engine rotational speed and the engine load. In the transport delay time setting process, the processing circuitry 11 calculates the first correction value by inputting the engine rotational speed and the engine load to the constant map.

When the vehicle 101 includes the recirculation passage 18, the amount of exhaust gas flowing through the exhaust passage 14 is reduced by the amount of exhaust gas recirculated to the intake passage 19. Further, when the vehicle 101 includes the recirculation passage 18, the transport delay time TL is increased by an amount corresponding to the recirculation of the exhaust gas to the intake passage 19.

The processing circuitry 11 corrects the reference transport delay time based on an EGR rate. The EGR rate is defined as the ratio of the amount of exhaust gas that has flowed into the intake passage 19 through the recirculation passage 18 to the sum of the amount of air drawn into the intake passage 19 and the amount of the exhaust gas. The term “EGR” in the EGR rate has the same meaning as “EGR” in the EGR valve 21.

For example, the processing circuitry 11 estimates the EGR rate based on the opening degree of the EGR valve 21 and the output value of the air flow meter 20. According to the EGR rate, the exhaust monitoring apparatus 100 stores, in the storage device 12, a constant table used to output a correction value that is based on the EGR rate. The correction value based on the EGR rate may be referred to as a second correction value. In the transport delay time setting process, the processing circuitry 11 calculates the second correction value by inputting the EGR rate to the constant table.

The processing circuitry 11 calculates the transport delay time TL by correcting the reference transport delay time using the first and second correction values. In the transport delay time setting process, the reference transport delay time is corrected in this manner to set the transport delay time TL.

As shown in FIG. 3, after executing the transport delay time setting process, the processing circuitry 11 executes the process of S12. In the process of S12, the processing circuitry 11 stores, in the storage device 12, the output value of the air flow meter 20 acquired at the start of the series of processes illustrated in FIG. 3. In the process of S12, the processing circuitry 11 stores the time at which the air flow meter 20 performed measurement in the storage device 12. In the process of S12, the processing circuitry 11 stores, in the storage device 12, the transport delay time TL set in the process of S11. Then, the processing circuitry 11 ends the series of processes illustrated in FIG. 3.

### Processes When the Output Value of the Nitrogen Oxide Sensor 17 is Acquired

FIG. 5 illustrates a series of processes executed by the exhaust monitoring apparatus 100. The series of processes illustrated in FIG. 5 is executed by the processing circuitry 11 in the electronic control unit 10. Upon acquiring an output value from the nitrogen oxide sensor 17, the processing circuitry 11 starts executing the series of processes illustrated in FIG. 5. In addition to the output value of the nitrogen oxide sensor 17, the processing circuitry 11 acquires information indicating the time at which the nitrogen oxide sensor 17 performed measurement.

### Outline of Search Process

Upon starting the series of processes illustrated in FIG. 5, the processing circuitry 11 first executes the process of S21. In the process of S21, the processing circuitry 11 executes a search process. In the search process, the processing circuitry 11 searches output values of the air flow meter 20, which are stored in the storage device 12, for the output value of the air flow meter 20 corresponding to the output value of the nitrogen oxide sensor 17. The output value of the air flow meter 20 corresponding to the output value of the nitrogen oxide sensor 17 is the output value of the air flow meter 20 that was output earlier, by the transport delay time TL, than the point in time at which the nitrogen oxide sensor 17 output the output value of the nitrogen oxide sensor 17 acquired by the processing circuitry 11 at the start of the processes shown in FIG. 5.

The outline of the search process will now be described below with reference to FIG. 6. FIG. 6 illustrates how the processing circuitry 11 searches the output value of the air flow meter 20 in the search process.

FIG. 6 illustrates two types of data, namely, data DA1 and data DA2.

The data DA1 indicates the output value from the nitrogen oxide sensor 17 acquired by the processing circuitry 11 at the start of the series of processes illustrated in FIG. 5. The data DA1 also indicates the time at which the nitrogen oxide sensor 17 performed measurement. In the data DA1, the output value of the nitrogen oxide sensor 17 is denoted by “OPn,” and the time at which the nitrogen oxide sensor 17 performed measurement is denoted by “PTn.” The time at which the nitrogen oxide sensor 17 performed measurement is hereinafter referred to as a nitrogen oxide sensor measurement time.

The data DA2 indicates a combination of the output value of the air flow meter 20, the time at which the air flow meter 20 performed measurement, and the transport delay time TL set by the processing circuitry 11. The time at which the air flow meter 20 performed measurement is hereinafter referred to as an air flow meter measurement time. The data DA2 indicates information stored in the storage device 12 in the process of step S12. In the search process, the processing circuitry 11 searches the data DA2 for the output value of the air flow meter 20 corresponding to the output value of the nitrogen oxide sensor 17 included in the data DA1.

In the data DA2, the output value of the air flow meter 20 is denoted by “OPa,” “OPb,” and “OPc.” In the data DA2, the air flow meter measurement time is denoted by “PTa,” “PTb,” and “PTc.” In the data DA2, the transport delay time TL is denoted by “TLa,” “TLb,” and “TLc.”

In the data DA2, the output value output by the air flow meter 20 through the measurement performed by the air flow meter 20 at time PTa is OPa. After the air flow meter 20 outputs OPa, the processing circuitry 11 that has acquired OPa sets the transport delay time TL to TLa in the transport delay time setting process. Hereinafter, a combination of OPa, PTa, and TLa is referred to as a combination Ga.

In the data DA2, the output value output by the air flow meter 20 through the measurement performed by the air flow meter 20 at time PTb is OPb. After the air flow meter 20 outputs OPb, the processing circuitry 11 that has acquired OPb sets the transport delay time TL to TLb in the transport delay time setting process. Hereinafter, a combination of OPb, PTb, and TLb is referred to as a combination Gb.

In the data DA2, the output value output by the air flow meter 20 through the measurement performed by the air flow meter 20 at time PTc is OPc. After the air flow meter 20 outputs OPc, the processing circuitry 11 that has acquired OPc sets the transport delay time TL to TLc in the transport delay time setting process. Hereinafter, a combination of OPc, PTc, and TLc is referred to as a combination Gc.

In the search process, the processing circuitry 11 performs search using the nitrogen oxide sensor measurement time in the data DA1, the air flow meter measurement time in the data DA2, and the transport delay time TL combined with the air flow meter measurement time. Specifically, the processing circuitry 11 compares the nitrogen oxide sensor measurement time with the time at which the transport delay time TL has elapsed from the air flow meter measurement time.

In the following description, the processing circuitry 11 searches the combinations Ga, Gb, and Gc for the output value of the air flow meter 20 corresponding to OPn.

In order to determine whether OPa, which is included in the combination Ga, is the output value of the air flow meter 20 corresponding to OPn, the processing circuitry 11 compares PTn with the time at which TLa has elapsed from PTa in the combination Ga.

When PTn is within a certain range of time with reference to the time at which TLa has elapsed from PTa, the processing circuitry 11 determines that OPa is the output value of the air flow meter 20 corresponding to OPn. When PTn is not within a certain range of time with reference to the time at which TLa has elapsed from PTa, the processing circuitry 11 determines that OPa is not the output value of the air flow meter 20 corresponding to OPn.

In the same manner, when determining that OPa, which is included in the combination Ga, is not the output value of the air flow meter 20 corresponding to OPn, the processing circuitry 11 determines whether OPb, which is included in the combination Gb, is the output value of the air flow meter 20 corresponding to OPn. That is, when PTn is within a certain range of time with reference to the time at which TLb has elapsed from PTb, the processing circuitry 11 determines that OPb is the output value of the air flow meter 20 corresponding to OPn. When PTn is not within a certain range of time with reference to the time at which TLb has elapsed from PTb, the processing circuitry 11 determines that OPb is not the output value of the air flow meter 20 corresponding to OPn.

When determining that OPb, which is included in the combination Gb, is not the output value of the air flow meter 20 corresponding to OPn, the processing circuitry 11 determines whether OPc, which is included in the combination Gc, is the output value of the air flow meter 20 corresponding to OPn in the same manner. That is, when PTn is within a certain range of time with reference to the time at which TLc has elapsed from PTc, the processing circuitry 11 determines that OPc is the output value of the air flow meter 20 corresponding to OPn. When PTn is not within a certain range of time based on the time at which TLc has elapsed from PTc, the processing circuitry 11 determines that OPc is not the output value of the air flow meter 20 corresponding to OPn.

In this manner, in the search process, the processing circuitry 11 searches the data DA2 for a combination that satisfies the condition that the nitrogen oxide sensor measurement time falls within a certain range of time with reference to the time at which the transport delay time TL has elapsed from the air flow meter measurement time. Upon finding a combination that satisfies such a condition in the data DA2, the processing circuitry 11 determines that the output value of the air flow meter 20 included in that combination is the output value of the air flow meter 20 corresponding to the output value of the nitrogen oxide sensor 17.

### Outline of Volume Estimation Process

As illustrated in FIG. 5, after executing the search process, the processing circuitry 11 advances the process to S22. In the process of S22, the processing circuitry 11 executes a volume estimation process. The volume estimation process estimates the volume of exhaust gas per unit time that has reached the portion where the nitrogen oxide sensor 17 is provided. The volume of exhaust gas per unit time that has reached the portion where the nitrogen oxide sensor 17 is provided is hereinafter referred to as an actual volumetric air flow rate.

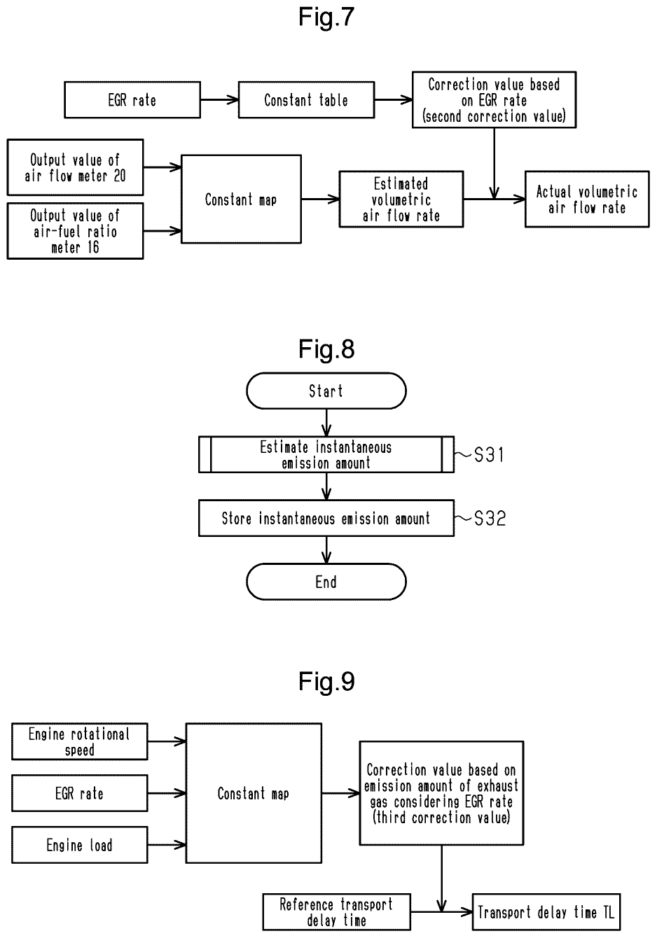

The outline of the volume estimation process will now be described with reference to FIG. 7. FIG. 7 illustrates how the processing circuitry 11 estimates the actual volumetric air flow rate in the volume estimation process.

The air drawn into the vehicle 101 is mixed with the fuel injected from the injector to form an air-fuel mixture. After combustion in the engine 13, the air-fuel mixture reaches the nitrogen oxide sensor 17 in the form of exhaust gas. As a result of combustion of the air-fuel mixture in the engine 13, the exhaust gas discharged from the combustion chamber has a greater volume than the intake air. The volume of exhaust gas discharged from the combustion chamber relates to a combination of the output value of the air flow meter 20 and the output value of the air-fuel ratio sensor 16.

The exhaust monitoring apparatus 100 stores, in the storage device 12, a constant map used to output the estimated volumetric air flow rate in accordance with a combination of the output value of the air flow meter 20 and the output value of the air-fuel ratio sensor 16. The estimated volumetric air flow rate represents the volume of exhaust gas discharged from the combustion chamber per unit time.

In the volume estimation process, the processing circuitry 11 estimates the estimated volumetric air flow rate from the output value of the air flow meter 20 and the output value of the air-fuel ratio sensor 16 based on the constant map. The processing circuitry 11 uses, as the output value of the air flow meter 20, the output value that was determined to correspond to the output value of the nitrogen oxide sensor 17 in the search process.

When the vehicle 101 includes the recirculation passage 18, the amount of exhaust gas flowing through the exhaust passage 14 is reduced by the amount of exhaust gas recirculated to the intake passage 19. When the vehicle 101 includes the recirculation passage 18, the volume of the exhaust gas that reaches the portion where the nitrogen oxide sensor 17 is installed is reduced by an amount corresponding to the recirculation of the exhaust gas to the intake passage 19.

The processing circuitry 11 corrects the estimated volumetric air flow rate based on the EGR rate. The exhaust monitoring apparatus 100 stores, in the storage device 12, a constant table used to output the second correction value according to the EGR rate. The second correction value output from the constant table shown in FIG. 4 is used to correct the reference transport delay time. The second correction value output from the constant table shown in FIG. 7 is used to correct the estimated volumetric air flow rate.

In the volume estimation process, the processing circuitry 11 estimates the EGR rate in the same manner as in the transport delay time setting process. In the volume estimation process, the processing circuitry 11 then calculates the second correction value by inputting the EGR rate to the constant table. In the volume estimation process, the processing circuitry 11 estimates the actual volumetric air flow rate by correcting the estimated volumetric air flow rate using the second correction value.

After executing the volume estimation process, the processing circuitry 11 ends the series of processes illustrated in FIG. 5.

### Process that Estimates the Instantaneous Discharge Amount

FIG. 8 illustrates a series of processes executed by the exhaust monitoring apparatus 100. The series of processes illustrated in FIG. 8 is executed by the processing circuitry 11 of the electronic control unit 10. After executing the series of processes illustrated in FIG. 5, the processing circuitry 11 starts executing the series of processes illustrated in FIG. 8.

Upon starting the series of processes illustrated in FIG. 8, the processing circuitry 11 first executes the process of S31. In the process of S31, the processing circuitry 11 estimates the instantaneous emission amount.

The processing circuitry 11 estimates the instantaneous emission amount based on the actual volumetric air flow rate, the output value of the nitrogen oxide sensor 17, the density of NOx, and a routine time. By using the following Equation (1), the processing circuitry 11 estimates the instantaneous emission amount, where W represents the instantaneous emission amount, EV represents the actual volumetric air flow rate, CN represents the output value of the nitrogen oxide sensor 17, DN represents the density of NOx, and RT represents the routine time.

W=EV×CN×DN×RT (1)

In Equation (1), CN represents the output value of the nitrogen oxide sensor 17 acquired by the processing circuitry 11 at the start of the series of processes illustrated in FIG. 5.

In Equation (1), EV is a value output through the process of S22 based on the output value of the air flow meter 20 corresponding to CN.

In Equation (1), DN represents, for example, a mass of NOx per liter at a standard state, assuming that the total amount of NOx is nitrogen dioxide.

In Equation (1), RT represents a cycle in which the nitrogen oxide sensor 17 outputs an output value.

As shown in FIG. 8, after estimating the instantaneous emission amount, the processing circuitry 11 advances the process to S32. In the process of S32, the processing circuitry 11 stores, in the storage device 12, the instantaneous emission amount output in the process of S31. Then, the processing circuitry 11 ends the series of processes illustrated in FIG. 8.

While the vehicle 101 is traveling, the exhaust monitoring apparatus 100 periodically estimates the instantaneous emission amount through the series of processes illustrated in FIGS. 3, 5, and 8, and stores the instantaneous emission amount in the storage device 12. This allows the exhaust monitoring apparatus 100 to estimate a total amount of NOx emitted from the vehicle 101 by accumulating the stored instantaneous emission amounts.

### Operation of the Present Embodiment

The air that has passed through the portion where the air flow meter 20 is provided requires a certain amount of time to form exhaust gas and then reach the nitrogen oxide sensor 17. Thus, if the output value of the air flow meter 20 and the output value of the nitrogen oxide sensor 17 that were output at the same time were used, the exhaust monitoring apparatus 100 could not accurately estimate the amount of NOx emitted from the vehicle 101.

In the present embodiment, the exhaust monitoring apparatus 100 estimates the emission amount of Nox considering the transport delay time TL for air from the portion where the air flow meter 20 is provided to reach the nitrogen oxide sensor 17.

### Advantages of the Present Embodiment

(1) The exhaust monitoring apparatus 100 accurately estimates the emission amount of NOx.
(2) The processing circuitry 11 sets the transport delay time TL based on the rotational speed of the engine 13 and the load of the engine 13.

The larger the amount of exhaust gas discharged from the combustion chamber of the engine 13, the higher the flow speed of the exhaust gas. The smaller the amount of exhaust gas discharged from the combustion chamber, the lower the flow speed of the exhaust gas. The amount of exhaust gas discharged from the combustion chamber affects the transport delay time TL. The amount of exhaust gas discharged from the combustion chamber is determined based on the engine rotational speed of the engine 13 and the load of the engine 13.

The exhaust monitoring apparatus 100 changes the transport delay time TL according to the rotational speed of the engine 13 and the load of the engine 13, thereby setting the transport delay time TL considering the amount of exhaust gas discharged from the combustion chamber. This enables the exhaust monitoring apparatus 100 to set the transport delay time TL with improved accuracy. Thus, the exhaust monitoring apparatus 100 estimates the emission amount of NOx more accurately.
(3) The vehicle 101 includes the recirculation passage 18, which recirculates exhaust gas to the intake passage 19. The processing circuitry 11 corrects the transport delay time TL based on the EGR rate. The EGR rate refers to the amount of exhaust gas that has flowed into the intake passage 19 through the recirculation passage 18 to the sum of the amount of air drawn into the intake passage 19 and the amount of the exhaust gas.

When exhaust gas is recirculated to the intake passage 19 through the recirculation passage 18, the amount of the exhaust gas flowing through the exhaust passage 14 is reduced by the amount of the recirculated exhaust gas. As a result, the transport delay time TL increases. The exhaust monitoring apparatus 100 corrects the transport delay time TL based on the EGR rate. This allows the exhaust monitoring apparatus 100 to estimate the emission amount of NOx more accurately.
(4) The exhaust monitoring apparatus 100 includes the air-fuel ratio sensor 16. The air-fuel ratio sensor 16 is disposed upstream of the exhaust purification device 15 in the exhaust passage 14. The air-fuel ratio sensor 16 measures the air-fuel ratio in the exhaust gas. The processing circuitry 11 estimates the volume of exhaust gas based on a past output value of the air flow meter 20 corresponding to the transport delay time TL and the output value of the air-fuel ratio sensor 16. The processing circuitry 11 estimates the emission amount of NOx based on the volume of exhaust gas, the output value of the nitrogen oxide sensor 17, and the density of NOx.

The volume of exhaust gas generated as a result of combustion of the air-fuel mixture in the engine 13 is greater than the volume of the air from which the exhaust gas was generated. The exhaust monitoring apparatus 100 estimates the volume of exhaust gas based on the output value of the air flow meter 20 and the air-fuel ratio. Further, the exhaust monitoring apparatus 100 estimates the emission amount of NOx based on the estimated volume of exhaust gas, the concentration of NOx detected by the nitrogen oxide sensor 17, and the density of the NOx. This allows the exhaust monitoring apparatus 100 to estimate the emission amount of NOx more accurately.
(5) The vehicle 101 includes the recirculation passage 18, which recirculates exhaust gas to the intake passage 19. The processing circuitry 11 corrects the estimated volume of exhaust gas based on the EGR rate.

When the exhaust gas is recirculated to the intake passage 19, the amount of the exhaust gas reaching the nitrogen oxide sensor 17 is reduced by the amount of the recirculated exhaust gas. When the vehicle 101 includes the recirculation passage 18, the actual volume of the exhaust gas reaching the nitrogen oxide sensor 17 is smaller than the volume estimated based on the output value of the air flow meter 20.

The exhaust monitoring apparatus 100 estimates the emission amount of NOx considering the amount of exhaust gas recirculated to the intake passage 19. This allows the exhaust monitoring apparatus 100 to estimate the emission amount of NOx more accurately.

### Modifications

The above-described embodiment may be modified as follows. The above-described embodiment and the following modifications can be combined as long as the combined modifications remain technically consistent with each other.

The vehicle 101 includes the recirculation passage 18. However, the vehicle 101 does not have to include the recirculation passage 18.

In the transport delay time setting process, the exhaust monitoring apparatus 100 corrects the transport delay time TL based on the EGR rate. However, in the transport delay time setting process, the exhaust monitoring apparatus 100 does not have to correct the transport delay time TL based on the EGR rate.

In the transport delay time setting process, the exhaust monitoring apparatus 100 corrects the reference transport delay time based on the amount of exhaust gas discharged from the combustion chamber. However, in the transport delay time setting process, the exhaust monitoring apparatus 100 does not have to correct the reference transport delay time based on the amount of exhaust gas discharged from the combustion chamber.

In the volume estimation process, the exhaust monitoring apparatus 100 corrects the estimated volumetric air flow rate based on the EGR rate. However, in the volume estimation process, the exhaust monitoring apparatus 100 does not have to correct the estimated volumetric air flow rate based on the EGR rate. In this case, in the volume estimation process, the estimated volumetric air flow rate is directly used as the actual volumetric air flow rate.

The exhaust monitoring apparatus 100 sets the transport delay time TL each time the series of processes illustrated in FIG. 3 is executed through the transport delay time setting process. The exhaust monitoring apparatus 100 may use a fixed transport delay time TL when estimating the emission amount of NOx.

The exhaust monitoring apparatus 100 estimates the instantaneous emission amount, which is the emission amount of NOx, based on the volume of exhaust gas, the output value of the nitrogen oxide sensor 17, the density of NOx, and the routine time. However, the exhaust monitoring apparatus 100 may estimate the emission amount of NOx in a manner different from that of the embodiment.

The exhaust monitoring apparatus 100 sets the transport delay time TL by correcting the reference transport delay time using the first and second correction values. However, the exhaust monitoring apparatus 100 may set the transport delay time TL in a manner different from that of the embodiment.

FIG. 9 illustrates how the processing circuitry 11 of the exhaust monitoring apparatus 100 according to a first modification sets the transport delay time TL during the transport delay time setting process. The processing circuitry 11 of the first modification sets the transport delay time TL in the manner shown in FIG. 9, instead of that shown in FIG. 4, in the process of S11 in FIG. 3.

The processing circuitry 11 of the first modification corrects the reference transport delay time based on the discharge amount of exhaust gas considering the EGR rate.

The amount of exhaust gas discharged from the combustion chamber affects the transport delay time TL. The amount of exhaust gas discharged from the combustion chamber is reduced by the amount of the exhaust gas recirculated to the intake passage 19 through the recirculation passage 18. The discharge amount of exhaust gas considering the EGR rate is defined as the exhaust discharge amount calculated by taking into account a reduction in the amount of exhaust gas discharged from the combustion chamber due to exhaust gas recirculation through the recirculation passage 18.

The exhaust monitoring apparatus 100 of the first modification stores, in the storage device 12, a constant map used to output a correction value that is based on the discharge amount of exhaust gas considering the EGR rate. The correction value based on the discharge amount of exhaust gas considering the EGR rate may be referred to as a third correction value. The constant map is used to output a correction value corresponding to a combination of the engine rotational speed, the engine load, and the EGR rate. As shown in FIG. 9, in the transport delay time setting process, the processing circuitry 11 of the first modification calculates the third correction value by inputting the engine rotational speed, the engine load, and the EGR rate to the constant map.

The processing circuitry 11 of the first modification corrects the reference transport delay time using the third correction value to calculate the transport delay time TL. In the transport delay time setting process of the first modification, the reference transport delay time is corrected in this manner to set the transport delay time TL.

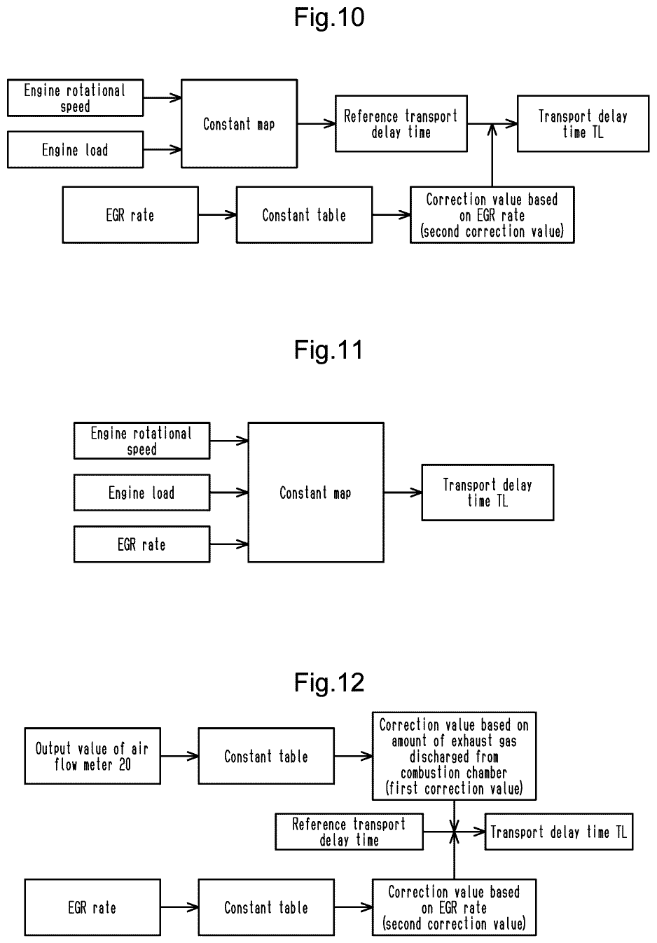

FIG. 10 illustrates how the processing circuitry 11 of the exhaust monitoring apparatus 100 according to a second modification sets the transport delay time TL during the transport delay time setting process. The processing circuitry 11 of the second modification sets the transport delay time TL in the manner shown in FIG. 10, instead of that shown in FIG. 4, in the process of S11 in FIG. 3.

The exhaust monitoring apparatus 100 of the second modification stores, in the storage device 12, a constant map used to output the reference transport delay time in accordance with a combination of the engine rotational speed and the engine load. In the transport delay time setting process, the processing circuitry 11 of the second modification calculates the reference transport delay time by inputting the engine rotational speed and the engine load to the constant map.

The processing circuitry 11 corrects the reference transport delay time based on the EGR rate. The exhaust monitoring apparatus 100 of the second modification stores, in the storage device 12, a constant table used to output the second correction value according to the EGR rate. In the transport delay time setting process, the processing circuitry 11 of the second modification calculates the second correction value by inputting the EGR rate to the constant table.

The processing circuitry 11 of the second modification calculates the transport delay time TL by correcting, using the second correction value, the reference transport delay time calculated based on the engine rotational speed and the engine load. In the transport delay time setting process of the second modification, the reference transport delay time is corrected in this manner to set the transport delay time TL.

FIG. 11 illustrates how the processing circuitry 11 of the exhaust monitoring apparatus 100 according to a third modification sets the transport delay time TL during the transport delay time setting process. The processing circuitry 11 of the third modification sets the transport delay time TL in the manner shown in FIG. 11, instead of that shown in FIG. 4, in the process of S11 in FIG. 3.

The exhaust monitoring apparatus 100 of the third modification stores, in the storage device 12, a constant map used to output the transport delay time TL in accordance with a combination of the engine rotational speed, the engine load, and the EGR rate. In the transport delay time setting process, the processing circuitry 11 calculates the transport delay time TL by inputting the engine rotational speed, the engine load, and the EGR rate to the constant map. In the transport delay time setting process of the third modification, the transport delay time TL is calculated in this manner to set the transport delay time TL.

In the above-described embodiment, as shown in FIG. 4, the exhaust monitoring apparatus 100 sets the transport delay time TL based on the engine rotational speed and the engine load. However, the exhaust monitoring apparatus 100 may set the transport delay time TL in a manner different from that of the embodiment. For example, the exhaust monitoring apparatus 100 may set the transport delay time TL based on the output value of the air flow meter 20.

FIG. 12 illustrates how the processing circuitry 11 of the exhaust monitoring apparatus 100 according to a fourth modification sets the transport delay time TL during the transport delay time setting process. The processing circuitry 11 of the fourth modification sets the transport delay time TL in the manner shown in FIG. 12, instead of that shown in FIG. 4, in the process of S11 in FIG. 3.

As shown in FIG. 12, the processing circuitry 11 of the fourth modification calculates the transport delay time TL by correcting the reference transport delay time.

The amount of exhaust gas discharged from the combustion chamber of the engine 13 affects the transport delay time TL. The processing circuitry 11 of the fourth modification corrects the reference transport delay time based on the amount of exhaust gas discharged from the combustion chamber.

The amount of exhaust gas discharged from the combustion chamber correlates with the amount of air drawn into the combustion chamber. The amount of exhaust gas discharged from the combustion chamber correlates with the output value of the air flow meter 20.

The exhaust monitoring apparatus 100 of the fourth modification stores, in the storage device 12, a constant table used to output the first correction value. The constant table is used to output the first correction value according to the output value of the air flow meter 20. In the transport delay time setting process, the processing circuitry 11 of the fourth modification calculates the first correction value by inputting the output value of the air flow meter 20 to the constant table. The output value of the air flow meter 20 input to the constant table by the processing circuitry 11 of the fourth modification is the output value of the air flow meter 20 acquired by the processing circuitry 11 of the fourth modification at the start of the series of processes illustrated in FIG. 3.

The processing circuitry 11 of the fourth modification corrects the reference transport delay time based on the EGR rate. The exhaust monitoring apparatus 100 of the fourth modification stores, in the storage device 12, a constant table used to output the second correction value according to the EGR rate. In the transport delay time setting process, the processing circuitry 11 of the fourth modification calculates the second correction value by inputting the EGR rate to the constant table.

As shown in FIG. 12, the processing circuitry 11 of the fourth modification calculates the transport delay time TL by correcting the reference transport delay time using the first and second correction values. In the transport delay time setting process of the fourth modification, the reference transport delay time is corrected in this manner to set the transport delay time TL.

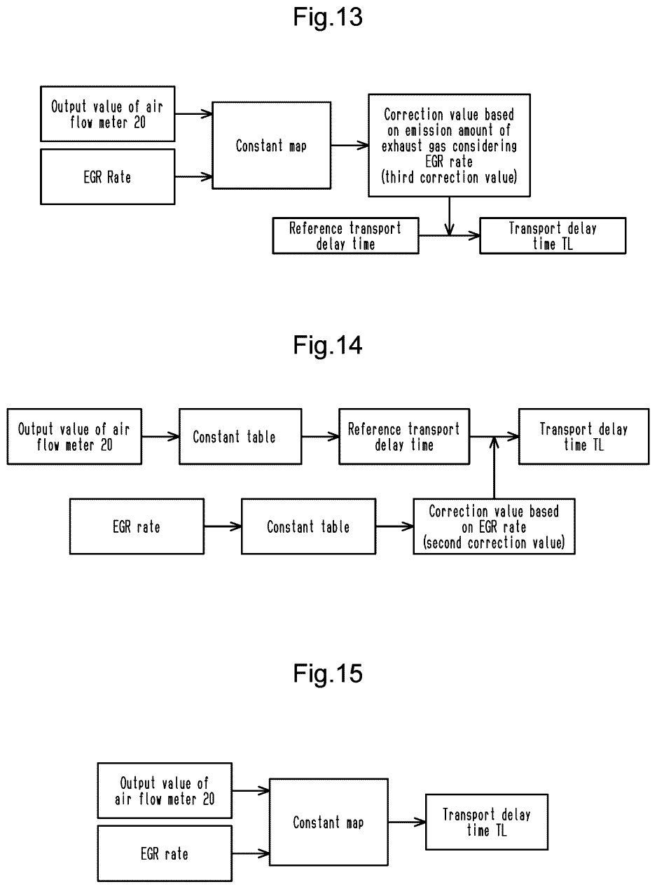

FIG. 13 illustrates how the processing circuitry 11 of the exhaust monitoring apparatus 100 according to a fifth modification sets the transport delay time TL during the transport delay time setting process. The processing circuitry 11 of the fifth modification sets the transport delay time TL in the manner shown in FIG. 13, instead of that shown in FIG. 4, in the process of S11 in FIG. 3.

As shown in FIG. 13, the processing circuitry 11 of the fifth modification corrects the reference transport delay time based on the discharge amount of exhaust gas considering the EGR rate.

The exhaust monitoring apparatus 100 of the fifth modification stores, in the storage device 12, a constant map used to output the third correction value. The constant map is used to output a correction value corresponding to a combination of the output value of the air flow meter 20 and the EGR rate. In the transport delay time setting process, the processing circuitry 11 of the fifth modification calculates the third correction value by inputting the output value of the air flow meter 20 and the EGR rate to the constant map. The output value of the air flow meter 20 input to the constant map by the processing circuitry 11 of the fifth modification is the output value of the air flow meter 20 acquired by the processing circuitry 11 of the fifth modification at the start of the series of processes illustrated in FIG. 3.

The processing circuitry 11 of the fifth modification calculates the transport delay time TL by correcting the reference transport delay time using the third correction value. In the transport delay time setting process of the fifth modification, the reference transport delay time is corrected in this manner to set the transport delay time TL.

FIG. 14 illustrates how the processing circuitry 11 of the exhaust monitoring apparatus 100 according to a sixth modification sets the transport delay time TL during the transport delay time setting process. The processing circuitry 11 of the sixth modification sets the transport delay time TL in the manner shown in FIG. 14, instead of that shown in FIG. 4, in the process of S11 in FIG. 3.

The exhaust monitoring apparatus 100 of the sixth modification stores, in the storage device 12, a constant table used to output the reference transport delay time in accordance with the output value of the air flow meter 20. In the transport delay time setting process, the processing circuitry 11 of the sixth modification calculates the reference transport delay time by inputting the output value of the air flow meter 20 to the constant table. The output value of the air flow meter 20 input to the constant table by the processing circuitry 11 of the sixth modification is the output value of the air flow meter 20 acquired by the processing circuitry 11 of the sixth modification at the start of the series of processing shown in FIG. 3.

The processing circuitry 11 of the sixth modification corrects the reference transport delay time based on the EGR rate. The exhaust monitoring apparatus 100 of the sixth modification stores, in the storage device 12, a constant table used to output the second correction value according to the EGR rate. In the transport delay time setting process, the processing circuitry 11 of the sixth modification calculates the second correction value by inputting the EGR rate to the constant table.

The processing circuitry 11 of the sixth modification calculates the transport delay time TL by correcting, using the second correction value, the reference transport delay time calculated based on the output value of the air flow meter 20. In the transport delay time setting process of the sixth modification, the reference transport delay time is corrected in this manner to set the transport delay time TL.

FIG. 15 illustrates how the processing circuitry 11 of the exhaust monitoring apparatus 100 according to a seventh modification sets the transport delay time TL during the transport delay time setting process. The processing circuitry 11 of the seventh modification sets the transport delay time TL in the manner shown in FIG. 15, instead of that shown in FIG. 4, in the process of S11 in FIG. 3.

The exhaust monitoring apparatus 100 of the seventh modification stores, in the storage device 12, a constant map used to output the transport delay time TL in accordance with a combination of the output value of the air flow meter 20 and the EGR rate. In the transport delay time setting process, the processing circuitry 11 of the seventh modification calculates the transport delay time TL by inputting the output value of the air flow meter 20 and the EGR rate to the constant map. The output value of the air flow meter 20 input to the constant map by the processing circuitry 11 of the seventh modification is the output value of the air flow meter 20 acquired by the processing circuitry 11 of the seventh modification at the start of the series of processing shown in FIG. 3. In the transport delay time setting process of the seventh modification, the transport delay time TL is calculated in this manner to set the transport delay time TL.

In this case, the processing circuitry 11 sets the transport delay time TL based on the output value of the air flow meter 20.

The larger the amount of exhaust gas discharged from the combustion chamber, the higher the flow speed of the exhaust gas. The smaller the amount of exhaust gas discharged from the combustion chamber, the lower the flow speed of the exhaust gas. That is, the amount of exhaust gas discharged from the combustion chamber affects the transport delay time TL. The amount of exhaust gas discharged from the combustion chamber correlates with the amount of air drawn into the combustion chamber. The amount of exhaust gas discharged from the combustion chamber correlates with the output value of the air flow meter 20, which measures the amount of air drawn into the combustion chamber.

The exhaust monitoring apparatus 100 sets the transport delay time TL considering the amount of exhaust gas discharged from the combustion chamber, by changing the transport delay time TL according to the output value of the air flow meter 20. This enables the exhaust monitoring apparatus 100 to set the transport delay time TL with improved accuracy, thereby estimating the emission amount of NOx more accurately.

Various changes in form and details may be made to the examples above without departing from the spirit and scope of the claims and their equivalents. The examples are for the sake of description only, and not for purposes of limitation. Descriptions of features in each example are to be considered as being applicable to similar features or aspects in other examples. Suitable results may be achieved if sequences are performed in a different order, and/or if components in a described system, architecture, device, or circuit are combined differently, and/or replaced or supplemented by other components or their equivalents. The scope of the disclosure is not defined by the detailed description, but by the claims and their equivalents. All variations within the scope of the claims and their equivalents are included in the disclosure.

What is claimed is:

1. An exhaust monitoring apparatus mounted on a vehicle including an exhaust purification device that is disposed in an exhaust passage of an engine, the exhaust purification device being configured to remove nitrogen oxides contained in exhaust gas, the exhaust monitoring apparatus comprising:
a nitrogen oxide sensor disposed downstream of the exhaust purification device in the exhaust passage and configured to measure a concentration of nitrogen oxides contained in exhaust gas;
an air flow meter disposed in an intake passage of the engine and configured to measure an amount of air flowing through the intake passage; and
processing circuitry configured to:
set a transport delay time, the transport delay time spanning from when air passes through a portion of the intake passage where the air flow meter is provided to when exhaust gas derived from the air that has passed through the air flow meter reaches the nitrogen oxide sensor; and
estimate an emission amount of nitrogen oxides based on an output value of the nitrogen oxide sensor and an output value of the air flow meter that was output earlier than a point in time at which the nitrogen oxide sensor output the output value by the transport delay time.

2. The exhaust monitoring apparatus according to claim 1, wherein
the processing circuitry is configured to set the transport delay time based on a rotational speed of the engine and a load of the engine.

3. The exhaust monitoring apparatus according to claim 1, wherein
the processing circuitry is configured to set the transport delay time based on the output value of the air flow meter.

4. The exhaust monitoring apparatus according to claim 1, wherein
the vehicle includes a recirculation passage configured to recirculate exhaust gas to the intake passage, and
the processing circuitry is configured to correct the transport delay time based on an EGR rate, the EGR rate being a ratio of an amount of exhaust gas that has flowed into the intake passage through the recirculation passage to a sum of an amount of air drawn into the intake passage and the amount of the exhaust gas.

5. The exhaust monitoring apparatus according to claim 1, comprising an air-fuel ratio sensor disposed upstream of the exhaust purification device in the exhaust passage and configured to measure an air-fuel ratio in exhaust gas, wherein
the processing circuitry is configured to:
estimate a volume of exhaust gas based on the output value of the air-fuel ratio sensor and the output value of the air flow meter that was output earlier than the point in time at which the nitrogen oxide sensor output the output value by the transport delay time; and
estimate an emission amount of nitrogen oxides based on the volume of the exhaust gas, the output value of the nitrogen oxide sensor, and a density of the nitrogen oxides.

6. The exhaust monitoring apparatus according to claim 5, wherein
the vehicle includes a recirculation passage configured to recirculate exhaust gas to the intake passage, and
the processing circuitry is configured to correct, based on an EGR rate, the estimated volume of the exhaust gas, the EGR rate being a ratio of an amount of exhaust gas that has flowed into the intake passage through the recirculation passage to a sum of an amount of air drawn into the intake passage and the amount of the exhaust gas.
